(19) United States
(12) Patent Application Publication
(10) Pub. No.: US 2002/0058049 A1
(43) Pub. Date: May 16, 2002

(54) Stable ophthalmic preparation

(76) Inventors: Michelle Pik-Han Wong, Duluth, GA (US); Shau-Fong Yen, Atlanta, GA (US); Mary Sou, Alpharetta, GA (US); Kasey Jon Minick, Alpharetta, GA (US)

Correspondence Address: THOMAS HOXIE, NOVARTIS CORPORATION, PATENT AND TRADEMARK DEPT, 564 MORRIS AVENUE, SUMMIT, NJ, 079011027

(21) Appl. No.: 09/952,294

(22) Filed: Sep. 14, 2001

Related U.S. Application Data
(60) Provisional application No. 60/232,316, filed on Sep. 14, 2000.

(51) Int. Cl. A61K 9/00; A61F 2/00

(52) U.S. Cl. 424/400

(57) ABSTRACT

The present invention provides a stable method for storing a pharmaceutical composition containing a prostaglandin-like-compound. The method has the step of storing the composition in a polypropylene container.

The invention relates to a stable ophthalmic preparation containing a prostaglandin-like-compound that may be used to treat glaucoma by reducing intraocular pressure. More specifically, the present invention relates to an aqueous preparation containing a prostaglandin-like-compound having increased stability in a polypropylene container, and thus, not requiring refrigeration.

Glaucoma, an eye disorder afflicting various mammals, including primates, is characterized by increased intraocular pressure (ocular hypertension). In humans, such ocular hypertension is caused by an imbalance between the rate of secretion of aqueous humor by the ciliary epithelium into the anterior and posterior chambers of the eyes and the rate of outflow or drainage of the aqueous humor from the anterior and posterior chambers, primarily via the canal of Schlemm. It is generally believed that obstruction of the aqueous humor drainage is the primary cause of the imbalance.

Chronic glaucoma typically results in slow, progressive loss of visual fields, and, if not controlled, ultimately in blindness. Different active compounds are available to treat glaucoma, including various prostaglandin-like-compounds. Prostaglandin-like-compounds are classified depending on the structure and substituents carried on the alicyclic ring of the prostanoic acid skeleton. Further classification is based on the number of unsaturated bonds in the side chain indicated by numerical subscripts after the generic type of a prostaglandin.

For illustration purposes, the term “prostaglandin-like-compound” as used hereinafter refers to a prostaglandin, a prostaglandin derivative or a prostaglandin analogue.

For example, U.S. Pat. No. 5,001,153 teaches a topical composition which contains a 13,14-dihydro-15-keto-prostaglandin that is suitable for treating glaucoma. U.S. Pat. No. 5,270,049 discloses 2-decarboxyl-2-aminoalkyl prostaglandin derivatives for managing glaucoma. U.S. Pat. No. 4,599,353 discloses an eicosanoid composition for treating glaucoma. Of the prostaglandin derivatives and analogues, lipid soluble prostaglandin derivatives and analogues appear to be particularly efficacious. Such lipid solubility permits more ready penetration of the protective layers of the primate eye and it has been found that smaller quantities of such compounds can be used than non-lipid soluble prostaglandin-like-compounds.

However, unless refrigerated, lipid soluble prostaglandin derivatives and analogues show unacceptable stability in standard low density polyethylene (LDPE) containers. (See 2000 Physician's Desk Reference for Ophthalmology-Xalatan® (a lipid soluble prostaglandin derivative) must be stored under refrigeration at 2° to 8° C.). The requirement that the ophthalmic preparation be refrigerated greatly reduces the availability of the treatment to those in less developed parts of the world. Furthermore, even where available, refrigeration of the preparation increases the cost of the treatment to the patient, and thus, further reduces its availability to those in need.

There therefore exists a need in the art for a method for storing such preparation over periods of time without refrigeration.

### SUMMARY OF THE INVENTION

The present invention, therefore, provides a stable pharmaceutical preparation containing a prostaglandin-like-compound, which is filled in an air-tight container made of polypropylene. Various prostaglandin-like-compounds are known in the art. For example, the above-mentioned U.S. Pat. Nos. 5,001,153; 5,270,049 and 4,599,353 provide such prostaglandin-like-compounds. The invention also provides a method for stably storing a pharmaceutical composition containing an active compound selected from a prostaglandin, a prostaglandin derivative, or a prostaglandin analogue. The method has the step of providing the pharmaceutical composition, especially an ophthalmic composition, in a container produced from polypropylene, which has a flexural modulus up to 170,000 pounds per square inch. The invention, in addition, provides a container for stably storing an ophthalmic composition containing prostaglandin, wherein the bottle is made from polypropylene. The bottle does not substantially adsorb the active compound even when the composition is not refrigerated over a period between one and 18 months. The term “substantially” as used herein indicates less than 3 wt %, preferably less than 2 wt %.

### DETAILED DISCUSSION

It has been surprisingly found that the stability of a preparation comprising a prostaglandin-like-compound, especially a lipid-soluble prostaglandin-like-compound, in a polypropylene bottle is increased to such a degree over the bottles currently used in the art, e.g., LDPE bottles, that refrigeration is no longer necessary, e.g., the preparation can be stored at room temperature. Thus, the ophthalmic preparation of the present invention has preferably a lipid-soluble prostaglandin-like-compound in a bottle-dropper assembly constructed from polypropylene having sufficient squeezeability to dispense drops by digital manipulation of the bottle by the user.

A typical bottle assembly has a plastic squeezable bottle, a nozzle tip or dropper which is snap fit into the bottle and a cap or closure which is threaded onto the bottle. Liquid is dispensed one drop at a time by squeezing the bottle so as to force liquid out the end of the nozzle tip. In accordance with the present invention, the bottle is produced from polypropylene having a modulus of elasticity and a flexural modulus for squeezing the cylindrical sidewall of the bottle with one's fingers to cause the liquid therein to pass through a passageway. Preferably, the polypropylene used in the bottle construction has a flexural modulus up to about 170,000 pounds per square inch (1200 MPa), more preferably a flexural modulus between about 100,000 psi (690 MPa) and about 160,000 psi (1100 MPa), as measured in accordance with ASTM D790. The invention also envisions a tube or bottle dropper assembly with a high enough squeezeability for dispensing an ophthalmic solution or gel by compressing the tube or bottle. Desirably, the nozzle tip and the cap are also made from polypropylene. In another embodiment of the present invention, the polymer for the container, e.g., bottle, may contain a minor amount of a polymer other than polypropylene. The polymer for the bottle may contain up to 20 wt %, more preferably up to 15 wt %, most preferably up to 5 wt %, of polyethylene.

While any prostaglandin-like-compound, especially lipid soluble prostaglandin-like-compounds, known in the art may be used in accordance with the present invention, the preferred prostaglandin-like-compound is unoprostone isopropyl ester (a lipid soluble prostaglandin analogue), which is commercially available from UENO Corporation.

As discussed above, a prostaglandin-like-compound is typically absorbed to the walls of the currently-used polyethylene, e.g., LDPE, bottles and the concentration thereof in the preparation lowers rapidly unless the preparation is refrigerated. However, the inventors have surprisingly found that a prostaglandin-like-compound, especially a lipid soluble prostaglandin-like-compound, is not significantly adsorbed to the wall of polypropylene containers. Thus, the preparation is stable for prolonged periods of time at room temperature. While any polypropylene having a flexural modulus up to 170,000 pounds per square inch is suitable for the present invention, the preferred polypropylene is REXENE PPM23M2, available commercially from Huntsman, Houston, Tex. This polypropylene has A flexural modulus of 145,000 pounds per square inch. Particularly suitable polypropylene For the present invention is random copolymer propylene, which contains up to about 5 wt %, Preferable up to about 3 wt %, more preferably up to about 2wt %, of ethylene.

The instant invention will be more fully understood by reference to the following illustrative, but non-limiting examples.

### EXAMPLE 1

0.15 wt % unoprostone isopropyl ester (a typical prostaglandin analogue) solution in an isotonic solution containing mannitol and edetate disodium is prepared. The solution also contains 0.015 wt % benzalkonium chloride as a preservative. The solution is placed in a polypropylene bottle and a polyethylene bottle, which bottles have 7.5 ml void volume, and 5 ml of the solution is placed in each bottle. The bottles have air-tight caps. The polypropylene bottle is molded from REXENE PP23M2 polypropylene and the polyethylene bottle is produced from ALATHON 2025 linear density polyethylene (LDPE), which is available from DuPont. The bottles are placed in controlled environment at 40° C. and ambient humidity, and the concentration of unoprostone is periodically measured using HPLC. The results are shown in Table 1.

As demonstrated in Table 1, the data show that the stability of this formulation is significantly improved when the preparation is packaged in polypropylene copolymer bottles, as opposed to the LDPE bottle currently used in the art.

TABLE 1
Stability of Unoprostone Solution in PP and LDPE Containers at 40° C.

| Time | | |
|---|---|---|
| (months) | % Active in LDPE Container | % Active in PP Container |
| 0 | 96.7 | 101.3 |
| 1 | 92.7 | 100.7 |
| 2 | 90.7 | 100.0 |
| 3 | 86.0 | 100.7 |
| 6 | 84.0 | 100.0 |

### EXAMPLE 2

Example 1 is repeated, except the Unoprostone samples are subjected to different storage temperatures and additional samples are placed in glass bottles with caps. In addition, different volumes of the sample solution are placed as indicated in Table 2.

As put forth in Table 2, the data show that product stored in LDPE with lower fill volumes result in a more significant decrease in the prostaglandin concentration than those in higher fill volumes. The samples stored in polypropylene bottles do not exhibit any significant decreases from the initial values.

TABLE 2
Stability of Unoprostone Isopropyl Ophthalmic Solution in PP, LDPE, and
Glass at Various Temperatures

| | | Initial | | | | | | |
|---|---|---|---|---|---|---|---|---|
| | | (% In | One Month | | | Two Months | | |
| Description | | UI) | 5° C. | 25° C. | 40° C. | 5° C. | 25° C. | 40° C. |
| PP | 1.5 | 98.0 | 97.3 | 97.6 | 99.2 | N/A | 98.6 | 100.3 |
| | ml | | | | | | | |
| | 3.0 | 97.7 | 97.5 | 97.2 | 97.0 | N/A | 97.9 | 98.2 |
| | ml | | | | | | | |
| | 5.5 | 97.6 | 97.9 | 98.5 | 97.9 | N/A | 98.6 | 98.8 |
| | ml | | | | | | | |
| LDPE | 1.5 | 95.8 | 95.9 | 90.5 | 83.9 | N/A | 89.4 | 83.4 |
| | ml | | | | | | | |
| | 3.0 | 95.8 | 96.3 | 94.3 | 90.3 | N/A | 91.7 | 88.4 |
| | ml | | | | | | | |
| | 5.5 | 97.5 | 97.0 | 95.1 | 92.7 | N/A | 94.5 | 93.2 |
| | ml | | | | | | | |
| Glass | 1.5 | 98.3 | N/A | 98.6 | 97.7 | N/A | N/A | 90.6 |
| | ml | | | | | | | |
| | 5.5 | 97.9 | N/A | 96.9 | 97.9 | N/A | N/A | 91.0 |
| | ml | | | | | | | |

### EXAMPLE 3

Example 1 is repeated except that three different temperatures are used to store the samples. As put forth in Table 3, the percent unoprostone isopropyl absorbed by polypropylene is much lower than LDPE. This is particularly significant in that the lower absorption at higher temperatures enables the preparation of the present invention to meet current FDA stability requirements without refrigeration.

TABLE 3

| Bottle Type | Storage Condition | % Unoprostone Absorbed |
|---|---|---|
| LDPE | 12 mo. at 5° C. | 3.11 |
| PP | 12 mo. at 5° C. | 0.00 |
| LDPE | 12 mo. at 25° C. | 4.76 |
| PP | 12 mo. at 25° C. | 0.98 |
| LDPE | 12 mo. at 30° C. | 5.70 |
| PP | 12 mo. at 30° C. | 1.98 |

### EXAMPLE 4

The unoprostone solution is placed in the polypropylene bottles, as described in Example 1, and placed in storage at 25° C. with 40% relative humidity. The stability over time of unoprostone in the polypropylene bottles is measured. The data in Table 4 show a surprising stability, where no measurable degradation in concentration of the prostaglandin derivative is detected over a period of eighteen months.

TABLE 4

| Time | % Unoprostone Isopropyl (Active) |
|---|---|
| Initial | 101.3% |
| 3 mo. | 101.3% |
| 6 mo. | 101.3% |
| 9 mo. | 101.3% |
| 18 mo. | 101.3% |

1. A stable pharmaceutical preparation within a bottle comprising polypropylene; wherein said pharmaceutical preparation comprises an active compound selected from a prostaglandin, a prostaglandin derivative, or a prostaglandin analogue.

2. A stable pharmaceutical preparation as claimed in claim 1, wherein said prostaglandin, prostaglandin derivative, or prostaglandin analogue is lipid soluble.

3. A stable pharmaceutical preparation as claimed in claim 1, wherein said active compound is unoprostone isopropyl ester.

4. A stable pharmaceutical preparation as claimed in claim 1, wherein said bottle does not substantially adsorb said active compound absent refrigeration over a period of time from 1 month to 18 months.

5. A stable pharmaceutical preparation as claimed in claim 4, wherein said bottle does not substantially adsorb said active compound at temperatures above 5° C.

6. A stable pharmaceutical preparation as claimed in claim 1, wherein said polypropylene has a flexural modulus up to 170,000 pounds per square inch.

7. A stable pharmaceutical preparation as claimed in claim 1, wherein said polypropylene has a flexural modulus between 100,000 and 160,000 pounds per square inch.

8. A stable pharmaceutical preparation as claimed in claim 6, wherein said polypropylene is a random copolymer of polypropylene and polyethylene.

9. A stable pharmaceutical preparation as claimed in claim 8, wherein said random copolymer comprises up to 5 wt % of polyethylene.

10. A stable pharmaceutical preparation as claimed in claim 6, wherein said bottle further comprises polyethylene.

11. A container for stably storing an ophthalmic composition comprising an active compound selected from a prostaglandin, a prostaglandin derivative, or a prostaglandin analogue, wherein said bottle comprises a polymer and said polymer consisting essentially of polypropylene.

12. The container of claim 11 wherein said polypropylene has a flexural modulus up to 170,000 pounds per square inch.

13. The container of claim 11 wherein said polypropylene has a flexural modulus between about 100,000 and about 160,000 pounds per square inch.

14. A method for stably storing a pharmaceutical composition comprising an active compound selected from a prostaglandin, a prostaglandin derivative, or a prostaglandin analogue, which method comprises the step of providing said composition in a container comprising polypropylene having a flexural modulus up to 170,000 pounds per square inch.

15. The method of claim 14 wherein said polypropylene has a flexural modulus between about 100,000 and about 160,000 pounds per square inch.

16. The method of claim 14 wherein said polypropylene is a random copolymer of propylene and ethylene, and wherein propylene comprises up to about 5 wt % of ethylene.

17. The method of claim 14 wherein said polypropylene comprises up to about 2 wt % of ethylene.

18. The method of claim 14 wherein said active compound is unoprostone isopropyl ester.
